(19) United States
(12) Patent Application Publication
(10) Pub. No.: US 2008/0171912 A1
(43) Pub. Date: Jul. 17, 2008

(54) Basal Blood pressure measuring method

(76) Inventor: Yi-Ta Chiang, Taipei (TW)

Correspondence Address: HDSL, 4331 STEVENS BATTLE LANE, FAIRFAX, VA, 22033, US

(21) Appl. No.: 11/553,995

(22) Filed: Oct. 28, 2006

(51) Int. Cl. A61B 5/00

(52) U.S. Cl. 600/300

(57) ABSTRACT

In a basal blood pressure measuring method, an examinee presses a basal blood pressure measuring key after the examinee gets out of bed in the morning to start a sphygmomanometer to measure the basal blood pressure A microprocessor in the sphygmomanometer displays the measured basal blood pressure and date on a display screen and saves the measured basal blood pressure into a basal blood pressure buffer of a memory automatically. When the examinee presses a basal blood pressure average key, the microprocessor in the sphygmomanometer computes and processes the basal blood pressure data in the memory and displays the basal blood pressure average of the current week or the past few weeks on the display screen to inform whether or not the examinee is a hypertension patient.

### BACKGROUND OF THE INVENTION

1. Field of the Invention

The present invention relates to a sphygmomanometer, and more particularly to a blood pressure measuring method provided for users to personally monitor their blood pressures.

2. Description of Prior Art

Hypertension (or high blood pressure) is a medical condition where the blood pressure exceeds a normal range; in other words, the systolic pressure exceeds 140 mm Hg or the diastolic pressure exceeds 90 mm Hg. The symptoms of hypertension are not obvious, but hypertension can cause gradual pathological changes to a patient's heart and blood vessels, and these pathological changes are dangerous sickness factors including stroke, heart disorder and kidney problem, etc and thus hypertension is generally called a “silent killer”.

There are many causes of hypertension including pigging-out, binge eating disorder, excessive exhaustion, anxiety, anger, irritant food, excessive fat, salty food and constipation. Some examinees may even have a high blood pressure measured at a hospital or a clinic but a normal blood pressure measured at home, and the examinees of this kind may get nervous easily and they may be affected by their environments more easily than other people. Therefore, doctors usually suggest the examinees to measure their blood pressure by a sphygmomanometer in the morning after the examinees get out of bed, and the measured blood pressure is used as a basis or reference for the basal blood pressure.

After an examinee measures a blood pressure by a sphygmomanometer available in the present market, the examinee's measured blood pressure is stored in the memory of the sphygmomanometer, such that when the examinee needs to know the average of the examinee's blood pressures, the examinee just needs to press an average key of the sphygmomanometer and then a microprocessor in the sphygmomanometer will compute the average of the measured blood pressures in the past few days or weeks. After the average of the blood pressures is computed, the average will be displayed on a display screen of the sphygmomanometer to inform the examinee or provide the information to doctors for reference. The accuracy of blood pressure measured by sphygmomanometers of this sort is a worthy subject for researches. A basal blood pressure is an examinee's blood pressure measured after the examinee gets out of bed in the morning. In addition to maintaining basic respiration and heartbeat, other metabolisms of a human body are situated at basal conditions, and the real condition of blood pressures can be obtained objectively by eliminating the factors that affect the blood pressures and these factors include an examinee's emotion, food, medication and work, etc. Therefore, if an examinee measures his/her blood pressure by a sphygmomanometer daily, and the blood pressure is not the measured basal blood pressure right after the examinee gets out of bed but measured at other time, then the measured blood pressure will be definitely affected by exercises, metabolisms, emotions and mental conditions. Therefore, the basal blood pressure has higher accuracy and reference values than the non-basal blood pressure. However, the general sphygmomanometers available in the market cannot identify and record the basal blood pressure value, so that the accuracy of basal blood pressure is not high enough.

### SUMMARY OF THE INVENTION

In view of the foregoing shortcomings of the prior art, the inventor of the present invention based on years of experience in the related industry to conduct experiments and modifications, and finally designed a basal blood pressure measuring method in accordance with the present invention.

Therefore, the present invention is to provide a sphygmomanometer that includes a measured basal blood pressure key for measuring an examinee's basal blood pressure within a period of time after the examinee gets out of bed in the morning.

The basal blood pressure measuring method of the invention includes a basal blood pressure measuring key on a sphygmomanometer, such that an examinee can press the basal blood pressure measuring key to start measuring a basal blood pressure by the sphygmomanometer, and a microprocessor in the sphygmomanometer displays the measured basal blood pressure and date on a display screen. In the meantime, the microprocessor automatically saves the measured basal blood pressure in a basal blood pressure buffer of a memory as the examinee's basal blood pressure value.

When the examinee presses a basal blood pressure average key, the microprocessor in the sphygmomanometer computes and processes a basal blood pressure data in the memory and displays a basal blood pressure average of the current day, the current week, or the past few weeks on the display screen to inform the examinee about the condition of the basal blood pressure average in the past few weeks or provide the basal blood pressure average to doctors for reference, so as to determine whether or not the examinee is a hypertension patient.

### BRIEF DESCRIPTION OF DRAWINGS

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 is a schematic view of an electronic sphygmomanometer of the present invention;

FIG. 2 is a schematic view of a memory for storing measured blood pressure values in accordance with the present invention;

FIG. 3 is a flow chart of measuring a basal blood pressure and a general blood pressure in accordance with the present invention; and

FIG. 4 is a flow chart of calling average measured basal blood pressure and general blood pressure in accordance with the present invention.

### DETAILED DESCRIPTION OF THE INVENTION

The technical characteristics, features and advantages of the present invention will become apparent in the following detailed description of the preferred embodiments with reference to the accompanying drawings. However, the drawings are provided for reference and illustration only and are not intended for limiting the scope of the invention.

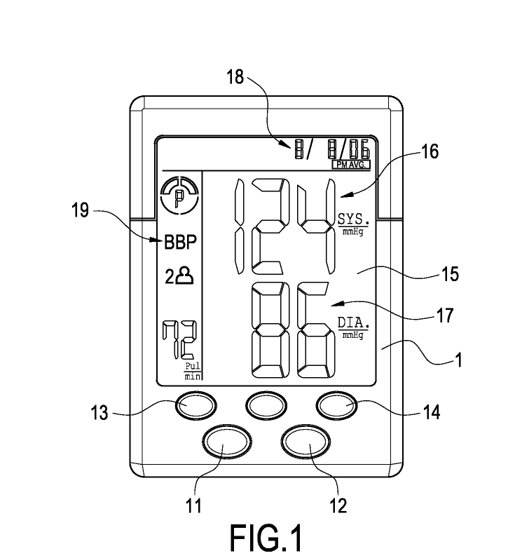

Referring to FIG. 1 to for a schematic view of an electronic sphygmomanometer of the present invention, a basal blood pressure measuring method of the invention includes a sphygmomanometer 1 having a basal blood pressure (BBP) measuring key 11, such that after an examinee presses the BBP measuring key 11, the sphygmomanometer 1 starts measuring the examinee's blood pressure within a period of time after the examinee gets out of bed in the morning as the examinee's basal blood pressure (BBP) reference value, so as to achieve the objective of self monitoring the examinee's blood pressure.

If an examine measures the blood pressure within a period of time after the examinee gets out of bed (such as 30 minutes or 1 hour after getting out of bed as specified by doctors), the examinee presses a BBP measuring key 11 on the sphygmomanometer 1 to start measuring a basal blood pressure by the sphygmomanometer 1, and the measured systolic pressure (mm Hg) 16, diastolic pressure (mm Hg) 17, date 18 and BBP symbol 19 are displayed on a display screen 15 of the sphygmomanometer 1. In the meantime, a microprocessor (not shown in the figure) of the sphygmomanometer 1 automatically saves the record of measured basal blood pressure into a basal blood pressure buffer of a memory (not shown in the figure) as the examinee's basal blood pressure value.

The examinee presses a general blood pressure measuring key 12 to measure a general blood pressure by the sphygmomanometer 1, and the measured systolic pressure (mm Hg) and diastolic pressure (mm Hg) and date are displayed on a display screen 15 of the sphygmomanometer 1. In the meantime, a microprocessor (not shown in the figure) in the sphygmomanometer 1 automatically saves the record of measured general blood pressure into a blood pressure buffer of a memory (not shown in the figure) as a record of the examinee's general blood pressure.

When the examinee presses a basal blood pressure average key 13 on the sphygmomanometer 1, the microprocessor in the sphygmomanometer 1 computes and processes a basal blood pressure data in a memory and displays a basal blood pressure average of the current day on the display screen 15. If the examinee presses the basal blood pressure average key 13, the basal blood pressure average of the current week will be displayed on the display screen 15. Therefore, pressing the basal blood pressure average key 13 once, the basal blood pressure averages of the current day, current week, previous week, previous two weeks and previous three weeks sequentially to inform the condition of the examinee's basal blood pressure average in recent few weeks or provide the basal blood pressure average to doctors for reference, so as to determine whether or not the examinee is a hypertension patient.

After the examinee presses a general blood pressure average key 14 of the sphygmomanometer 1, the microprocessor in the sphygmomanometer 1 computes and processes the general blood pressure data in memory and displays the general blood pressure average of the current day on the display screen 15. If the examinee presses the general blood pressure average key 14 once again, then the general blood pressure average of the current week will be displayed on the display screen 15. Therefore, the averages of general blood pressures of the current day, current week, previous week, previous two weeks and previous three weeks will be displayed sequentially when the general blood pressure average key 14 is pressed each time to inform the examinee about the conditions of the general blood pressure averages of the recent few weeks or provide the conditions of the general blood pressure averages of the recent few weeks to doctors for their reference.

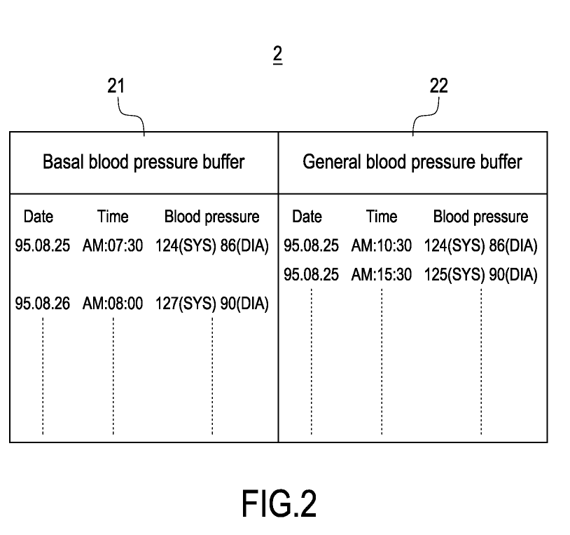

Referring to FIG. 2 for a schematic view of a memory for storing measured blood pressure values in accordance with the present invention, an examinee measures a basal blood pressure by a sphygmomanometer 1, and a microprocessor (not shown in the figure) in the sphygmomanometer 1 automatically stores the record of basal blood pressure into a basal blood pressure buffer 21 of the memory 2 as the examinee's basal blood pressure reference value.

After the examinee measures the general blood pressure, a microprocessor (not shown in the figure) of the sphygmomanometer 1 automatically saves the record of the measured blood pressure in a general blood pressure buffer 22 of the memory 2 as the examinee's general blood pressure reference value.

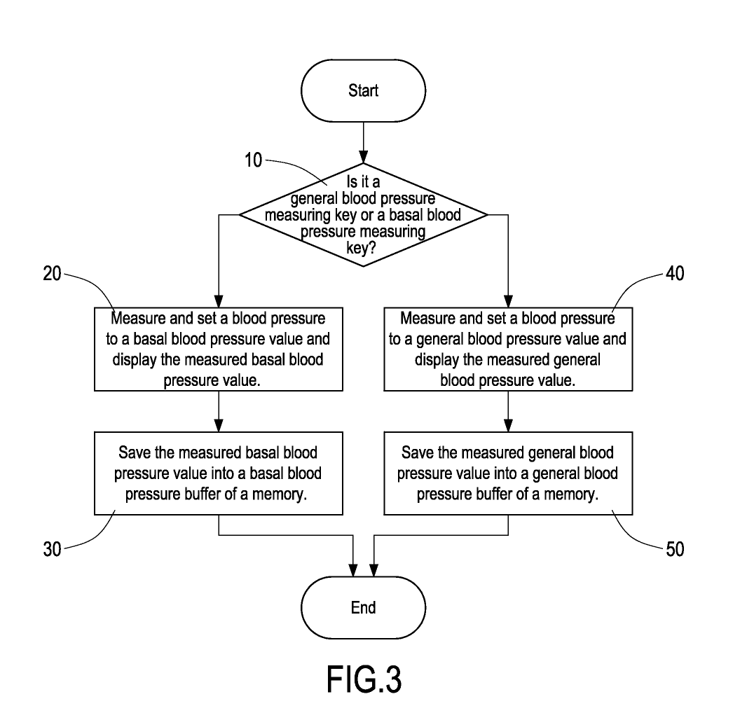

Referring to FIG. 3 to a flow chart of measuring a basal blood pressure and a general blood pressure in accordance with the present invention, when the examinee measures a blood pressure within a period of time after the examinee gets out of bed, a microprocessor in a sphygmomanometer 1 determines whether or not a general blood pressure measuring key or a basal blood pressure measuring key is pressed (as shown in Step 10). If it is determined that the basal blood pressure measuring key 11 is pressed, the microprocessor in the sphygmomanometer 1 sets the record of blood pressure as a basal blood pressure measured value and displays the basal blood pressure value (as shown in Step 20) and saves the measured basal blood pressure in a basal blood pressure buffer of the memory (as shown in Step 30), and then ends measuring the blood pressure.

When an examinee measures a blood pressure, the microprocessor in the sphygmomanometer 1 determines whether or not a general blood pressure measuring key is pressed (as shown in FIG. 10), the microprocessor in the sphygmomanometer 1 sets the record of blood pressure as a general blood pressure measured value and display the general blood pressure value (as shown in FIG. 40), and saves the general blood pressure measured value in a general blood pressure buffer (as shown in Step 50) of the memory, and ends measuring blood pressure.

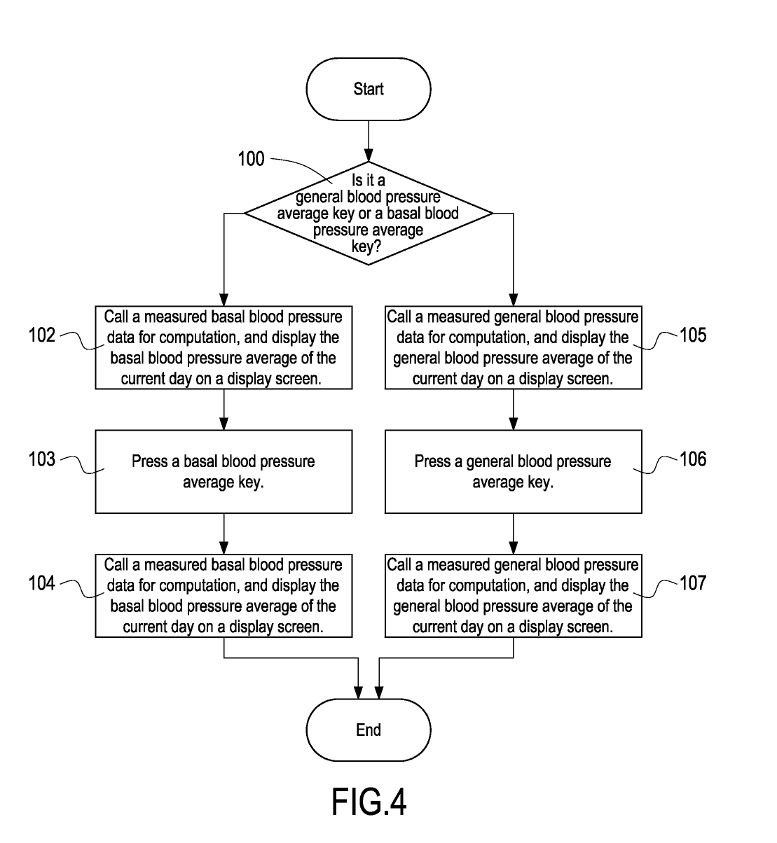

Referring to FIG. 4 for a flow chart of calling the measured average values of basal blood pressures and general blood pressures, when it determines whether or not the basal blood pressure average key 13 on the sphygmomanometer 1 is pressed (as shown in Step 100), and the microprocessor calls the measured basal blood pressure data from the memory for computing and processing and displaying the basal blood pressure average value of the current display screen 15 (as shown in Step 102). After the basal blood pressure average key 13 is pressed again (as shown as Step 103), the microprocessor calls the basal blood pressure measured data from the memory to compute and display the basal blood pressure average of the current week (previous week, previous two weeks or previous three weeks) on the display screen 15 (as shown in Step 104), and ends the procedure.

If a general blood pressure average key 14 of the sphygmomanometer 1 is determined to be pressed (as shown in Step 100), the microprocessor calls and computes the general blood pressure measured data of the current day from the memory, and displays the general blood pressure average on the display screen 15 (as shown in Step 105). After the general blood pressure average key 14 is pressed again (as shown in Step 106), the microprocessor measures a general blood pressure from the memory and displays the general blood pressure average of the current week (previous week, previous two weeks, and pervious three weeks) on the display screen 15 (as shown in Step 107), and ends the procedure.

The present invention is illustrated with reference to the preferred embodiment and not intended to limit the patent scope of the present invention. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. A basal blood pressure measuring method, for using a sphygmomanometer to measure and display a basal blood pressure of an examinee after an examinee has gotten out of bed for a while in the morning, and said method comprising the steps of:
(a) the sphygmomanometer having a basal blood pressure measuring key;
(b) measuring the basal blood pressure by pressing the basal blood pressure key;
(c) displaying the measured basal blood pressure and date on a display screen of the sphygmomanometer; and
(d) a microprocessor in the sphygmomanometer storing a record of measured basal blood pressure automatically into a memory.

2. The basal blood pressure measuring method of claim 1, wherein the value of basal blood pressure measured in Step (c) is stored in a basal blood pressure buffer of the memory.

3. The basal blood pressure measuring method of claim 1, wherein the sphygmomanometer has a basal blood pressure average key.

4. The basal blood pressure measuring method of claim 1, wherein the basal blood pressure average key is pressed to compute a basal blood pressure data by a microprocessor in the sphygmomanometer and display an average basal blood pressure of today, the current week, or previous few weeks on a display screen.

5. The basal blood pressure measuring method of claim 4, wherein the basal blood pressure average key is pressed to display a basal blood pressure symbol on the sphygmomanometer.
